(19) United States
(12) Patent Application Publication
(10) Pub. No.: US 2007/0265346 A1
(43) Pub. Date: Nov. 15, 2007

(54) Combination therapy for the treatment and improvement of scars

(76) Inventor: Anita Mehta, Plainfield, IL (US)

Correspondence Address: BRINKS HOFER GILSON & LIONE, P.O. BOX 10395, CHICAGO, IL, 60610, US

(21) Appl. No.: 11/786,085

(22) Filed: Apr. 10, 2007

Related U.S. Application Data
(60) Provisional application No. 60/791,310, filed on Apr. 11, 2006.

(51) Int. Cl. A61K 8/28; A61K 8/27; A61K 31/22; A61K 31/137

(52) U.S. Cl. 514/609; 514/546; 424/67; 514/649; 514/738

(57) ABSTRACT

The present invention is a composition, methods of using that composition and kits including that composition, useful for reducing the size and improving the appearance of a closed wound wherein the composition comprises a therapeutically effective amount of a hydrophilic or hydrophobic carrier (or a mixture thereof), at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) penetration enhancers; (j) antioxidants; (k) antipuritic agents; (l) fibrinolytic agents; (j) immunomodulators; (m) transcription modulating agents; (n) surface modulating agents; (o) growth factor inhibitors; and (p) anti-proliferative agents.

This application claims priority to U.S. Provisional Patent Application Ser. No. 60/791,310, filed Apr. 11, 2006.

### BACKGROUND OF THE INVENTION

Although scar formation and remodeling are essential processes in skin wound healing, disorders of excess scar formation, such as hypertrophic scars and keloids, remain a common clinical problem. A hypertrophic scar is an excessive wound scar which is thick and raised, having grown in size beyond that required for normal wound healing. A hypertrophic scar stays essentially within the boundaries of the original injury. A keloid is a raised scar that exceeds the boundaries of the initial injury, and is rarely corrected by surgical intervention.

The changing patterns of the connective tissue matrix during repair following injury require a delicate balance between synthesis and degradation of collagen and proteoglycans. Under normal circumstances this balance is maintained, while in many diseased states it is altered, leading to an excessive deposition of collagen, to a loss of functional tissue, or to disfigurement. With hypertrophic scars and keloids, the biosynthetic phase continues longer than necessary to repair the wound. In order to maintain nutrient supply in hypertrophic scars and keloids scars, vascular in-growth occurs, resulting in large, highly vascularized scars which are unsightly and can be disabling.

Existing therapy for hypertrophic scars and keloids includes surgery, mechanical pressure, steroids, x-ray irradiation and cryotherapy. There are many disadvantages associated with each of these methods. Surgical removal of the scar tissues is often incomplete and can result in further development of hypertrophic scars and keloids at the incision and suture points. Steroid treatments are unpredictable and often result in depigmentation of the skin. X-ray therapy is the only predictable effective treatment to date; however, because of its potential for causing cancer, it is not generally recommended or accepted. The most common approach to controlling scar, and in particular excessive scar formation, is to apply pressure, which appears to be somewhat effective in many instances. This treatment has limited application, generally based on the size and location of the scar tissue on the body. Other commonly used treatments are application of Vitamin E and corticosteroids. However, each of these agents is believed to interfere with collagen synthesis and promote collagen degradation.

Another existing therapy for scars involves achieving the right balance between the activity and inhibition of matrix metalloproteinases (MMPS). MMPs are a family of zinc dependent extracellular endoproteinases involved in scar formation. Inhibition of MMPs plays a role in regulating the cell-matrix interactions associated with a variety of physiological processes including tissue remodeling. Therefore, a disturbance in the activity or inhibition of MMPs may result in cellular dysfunction and can lead to excessive scar formation.

Thus, there is a need in the art for efficient treatment and prevention options for scars, as demonstrated by the Scar Evaluation and Management Recommendations published in the European Tissue Repair Society Bulletin, page 33-42, 2005, and Novel Opportunities in the Treatment and Prevention of scarring, A review by Brain Berman, JCMS, 2005, which are incorporated herein by reference. Such a treatment should focus on treating cellular dysfunction that occurs at all levels and phases of tissue remodeling.

### SUMMARY OF THE INVENTION

The present invention relates to methods and compositions for improving the appearance and/or reducing the size of a closed wound, which may be a scar. For example, a closed wound may be a hypertrophic scar, keloid, Dupuytren's contracture, fibrotic scar, or a reactive scar.

Accordingly, the present invention relates to methods for reducing the size or improving the appearance of a closed wound comprising administering to an individual having a closed wound or scar a therapeutically effective amount of a composition comprised of a hydrophilic or hydrophobic carrier (or a mixture of the same), wherein the composition comprises at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agent; (f) tissue remodeling correcting agents; (g) antimicrobial agent; (h)modulators of deposition of extra cellular matrix; (i) drug penetration/permeation enhancer agent; (j) antioxidative agent; (k) antipuritic agent; (l) fibrinolytic agents; (j) immunomodulators; (m) transcription modulating agents; (n) surface modulating agents; (o) growth factor inhibitors; or (p) anti-proliferative agents. In one example, the MMP modulator is verapamil.

The present invention also provides a medical device for treating the condition of excessive scar formation comprising a hydrophilic or hydrophobic carrier (or a mixture of the same), wherein the composition comprises at least one matrix metalloproteinase (MMP) modulator in combination with one or more of the following pharmaceutically active agents: a) cell cycle modulators; (b) inflammatory event modulators; (c) angiogenesis event modulators; (d) fibroblast migration agents; (e) fibroblast proliferation agent; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents. In one example, the MMP modulator used in the medical device of the present invention is verapamil. In a further example, a medical device of the present invention may be adapted for implantation or insertion in the coronary vasculature, peripheral vasculature, esophagus, colon, biliary tract, brain or liver of a patient.

The present invention also includes a method of simultaneous administration of the above-identified composition or medical device in combination with a polyethylene glycol material and/or an anti-irritant, for example, diphenhydramine, to reduce skin irritation.

In one embodiment, a method of the present invention includes contacting a closed wound with a thermal insulating material that elevates the surface temperature of the closed wound, wherein the thermal insulating material includes an effective amount of a composition comprising a hydrophilic or hydrophobic carrier (or a mixture of the same), and at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents. In one example, the MMP modulator is verapamil.

In another embodiment, the thermal insulating material may also include a deodorant agent to reduce surface bacteria and odor formation. The thermal insulating material is allowed to remain in contact with the closed wound for a period of time sufficient to allow a noticeable improvement in the size and appearance of the closed wound.

In yet another embodiment, a method for improving the appearance and/or reducing the size of a closed wound comprises contacting the closed wound with a thermal insulating material that elevates the surface temperature of the closed wound wherein the thermal insulating material includes an effective amount of a composition comprised of a hydrophilic or hydrophobic carrier (or a mixture of the same), wherein the composition comprises at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents. In this example, the thermal insulating material is allowed to remain in contact with the closed wound for a period of time sufficient to allow a noticeable improvement in the appearance of the closed wound and/or a reduction in the size of the closed wound. In one example, the MMP modulator is verapamil.

In another embodiment, the thermal insulating material described in the above paragraph may also contain an anti-irritant substance.

In yet another embodiment, the thermal insulating material may be a hydrogel and the hydrogel may contain an effective amount of salicylic acid or derivatives/analogues thereof. In this example, the hydrogel is allowed to remain in contact with the closed wound for a period of time sufficient to bring about an improvement in appearance and/or a reduction in the size of the closed wound.

In a further embodiment, the present invention is a composition comprising a PEG material in combination with an effective amount of at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents. In one example, the MMP modulator is verapamil.

In another embodiment, the present invention is a method of improving the appearance or reducing the size of a closed wound comprising contacting a closed wound with a PEG material that includes an effective amount of at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents. In one example, the MMP modulator is verapamil.

The PEG material also may include a deodorant agent and/or an anti-irritant. The PEG material is allowed to remain in contact with the closed wound for a period of time sufficient to allow a noticeable improvement in the size and appearance of the closed wound.

In a further embodiment, a method for improving the appearance and/or reducing the size of a closed wound comprises contacting the closed wound with a PEG material that includes an effective amount of a composition comprised of a hydrophilic or hydrophobic carrier (or a mixture of the same), wherein the composition comprises at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents. In one example, the MMP modulator is verapamil.

In this example, the PEG material is allowed to remain in contact with the closed wound for a period of time sufficient to allow a noticeable improvement in the appearance of the closed wound and/or a reduction in the size of the closed wound.

Yet another embodiment of the invention includes the use, for the manufacture of a medicament for preventing or treating a condition caused by the appearance of a closed wound, such as a hypertrophic or keloid scar, of an effective amount of a composition comprised of a hydrophilic or hydrophobic carrier (or a mixture of the same), wherein the composition comprises at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents. In one example, the MMP modulator is verapamil.

In one embodiment, this medicament is combined with a thermal insulating material or a PEG material.

In another embodiment the invention comprises a kit for use in improving the appearance and/or reducing the size of a closed wound. A kit according to an embodiment of the invention may include a composition comprised of a hydrophilic or hydrophobic carrier (or a mixture of the same), wherein the composition comprises at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents, and a hydrogel or a PEG material. In one example, the MMP modulator is verapamil.

In another embodiment, a kit may comprise a hydrogel or a PEG material that includes a composition comprised of a hydrophilic or hydrophobic carrier (or a mixture of the same), wherein the composition comprises at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents. In one example, the MMP modulator is verapamil.

### DETAILED DESCRIPTION OF THE INVENTION

It is to be understood that the present invention is not limited to the particular compositions, methodologies or protocols described herein. Further, unless defined otherwise, all technical and scientific terms used herein have the same meaning as commonly understood by one of ordinary skill in the art to which this invention belongs. It is also to be understood that the terminology used herein is for the purpose of describing particular embodiments only, and is not intended to limit the scope of the present invention, which will be limited only by the claims.

The present invention relates to methods and compositions for improving the appearance and/or reducing the size of a closed wound or a scar, and for reducing scar irritation. Treatment or prevention of an excessive scarring disorder in a mammal in need of such treatment or prevention is provided by methods and combinations using two or more components with at least one component being a matrix metalloproteinase (MMP) inhibitor.

The compositions and methods according to embodiments of the invention can be used on any vertebrate with skin. Examples of such vertebrates include mammals (for example, human, bovine, porcine, canine, feline) and avian.

The compositions of the present invention show improvement of a scar by reducing scar redness, thickness, tenderness, pain, symptoms of pruritus, firmness, hyperpigmentation and/or hypopigmentation.

As used herein, the terms “closed wound” or “scar” refer to a wound or a wound surface that is closed by regrowth of an epithelial barrier. A wound is “closed” after an open wound has been re-epithelialized. A wound is typically closed within 48-72 hours after injury. Closed wounds can result in the formation of a scar, which is never an exact replacement of the original tissue. Scar tissue is less elastic than the undamaged tissue and has surface and contour irregularities. As used herein, the term “affected area of skin” may also be used to refer to either a “closed wound” or a “scar.”

In one embodiment, a closed wound is an area of skin that has pain, tingling, burning, and/or itching. In another embodiment, a closed wound is a scar. In another embodiment, a scar is an area of skin that has pain, tingling, burning, itching, discoloration, surface irregularities, and/or an erratic accumulation of fibrous tissue.

A closed wound may result from any of a number of types of skin traumas such as laceration, avulsion, burn, surgery, infection, chemical facial peel, and accident. An open wound closes by regrowth of an epithelial barrier, the regrowth replacing some of the normal tissue which had been destroyed by trauma. Sometimes, in the closed wound or scar, excessive and disfiguring deposits of fibrous tissue having an erratic accumulation of collagen occur.

Normally, wound healing is a continuous process extending over a one-to-two-year period. Conceptually, the wound healing process may be divided into three phases.

The first phase is an intensely degradative phase called the inflammatory stage. It occurs immediately after injury and provides a means to remove the damaged tissues and foreign matter from the wound as well as regain immunological control over invading skin surface microbes. This phase lasts approximately one week when the wound is immediately closed, for example in a surgical incision. However, the level of the inflammatory response continues at elevated levels in open wounds until the wound surface is closed by regrowth of the epithelial barrier. If wound inflammation is prolonged or more intense, excessive scarring, called hypertrophic scars, usually appear.

Inflammation, or an “inflammatory response”, is the net result of interconnected physiological events, including increased vascular permeability, fluid accumulation, and the migration of a changing population of inflammatory cells into an inflamed area. The clinical manifestations of inflammation include swelling, increased local temperature, erythema, and pain. The inflammatory response can be triggered by any of a number of causative factors, including certain bacteria, radiation, hypersensitivity to chemical agents, and the like. The inflammatory response is generally believed to be a primary defense mechanism in the body, but, unchecked, can become excessive resulting in functional impairment.

The second stage of wound healing typically occurs 2-3 days later and typically lasts about 3 weeks. This stage may be referred to as the proliferation and matrix synthesis stage. During this stage fibroblasts from the surrounding tissue invade the wound and proliferate. The fibroblasts in the wound proliferate and actively produce macromolecules, such as collagen and proteoglycans, which are secreted into the extracellular matrix. Fibroblast activity is driven by the chemical signals produced by inflammation. The newly-synthesized collagen fibrils are cross-linked by lysyl oxidase and provide structural integrity to the wound. During this stage, fibroblasts also contract the intact collagen in order to reduce the surface area of the wound.

In the third and final stage, called the remodeling stage, the previously constructed and randomly organized matrix is remodeled into an organized structure which is highly cross-linked and aligned to maximize mechanical strength. Natural skin wrinkles (relaxed skin tension lines) which align themselves in the direction of mechanical tension and become permanent on the face over time are a common manifestation of this control process.

The end result of mammalian wound healing is scar formation. Scars are not an exact replacement for undamaged tissue. Skin scars are generally less elastic, creating contour irregularities; color changes and maybe painful if they entrap nerves. Control of dermal scarring is one of the most important objectives in the management of trauma particularly burn trauma. Minimizing dermal scarring and may lead to optimum post-traumatic functional and aesthetic recovery.

One such scar which can result from an overproduction of collagen and excess deposition of scar tissue is a hypertrophic scar. As used herein, the term “hypertrophic scar” includes a scar characterized by thick, raised scar tissue that stays essentially within the boundaries of the original injury. Hypertrophic scars contain characteristic nodules, and result from a full-thickness injury, such as a surgical incision on skin. These scars can cause problems such as aesthetic deformity and severe limitation of motion. For example, an excessive post-operative scar can develop as a result of “over-healing” or hypertrophic healing of a post-operative site.

Hypertrophic scars generally result from an over-production of cells, collagen and proteoglycan [Linares, H. A. et al., Plast. Reconst. Surg., 62:589 (1978); Linares, H. A., Plast. Reconstr. Surg., 818-820 (1983)]. These scars more frequently occur among children and adolescents, suggesting that growth factors may influence the development of this type of scar.

Hypertrophic scars are especially common in patients who have burns or wounds that are allowed to remain open for more than a few weeks. These scars, by definition, exceed normal wound healing, causing problems that range from aesthetic deformity to severe limitation of motion In these scars, the over-production and compaction of collagen and proteoglycans [Shetlar, M. R. et al., Burns 4:14 (1977)] exceeds the proliferation of cells. These histological observations suggest that the lesions result from loss of the normal control mechanisms which regulate the synthesis of extracellular matrix during wound healing [Shetlar. M. R. et al., Burns 4:14 (1977)].

Hypertrophic scars are more common on the anterior surfaces of the neck, the shoulder, the chest wall and, in general, the flexor surfaces of the extremities. While some hypertrophic scars will spontaneously resolve within a few years, in many instances, especially in the locations mentioned above, they persist indefinitely. Because these scars are so common, particularly in burns or wounds that heal by secondary intention, their management represents a major unsolved clinical problem.

Another type of scar in which there is an excess deposition of scar tissue is called a “reactive scar.” As the term is used herein, a reactive scar is a normal, healed scar which, through mechanical disruption such as scratching or other irritation, is actively producing a hypertrophic tissue response.

Excessive scar deposition also occurs in a “fibrotic scar.” As the term is used herein, a fibrotic scar is an accumulation of irritated fibrotic tissue at the site of a healed injury which may or may not have involved an observable wound.

Another type of scar that can result from an excess deposition of scar tissue is a keloid. As used herein, the term “keloid” includes a scar characterized by thick, raised scar tissue that exceeds the initial boundaries of the trauma and that lacks nodules. In contrast to hypertrophic scars, keloids proliferate beyond the wound edges, can result from superficial injuries, and are rarely treated successfully by surgery. Keloids frequently develop after burns, particularly where the skin is under tension, such as on the breastbone.

Although appearing similar, keloids and hypertrophic scars differ considerably. The former is genetically based with both autosomal dominant and autosomal recessive modes of transmission reported (Roseborough et al., J. Natl. Med. Assoc. 2004, 96(1), 1-9).The basis for the genetic differences has been shown through several investigations describing the aberrant behavior of keloid fibroblasts. It has also resulted from reports of keloids exhibiting abnormal regulation of apoptosis to abnormally producing collagen, fibronectin, and proteoglycans with atypical responses to metabolic regulators.

Unlike hypertrophic scars, keloids may occur with only minor insults to the skin. Conversely, hypertrophic scars are usually the result of injury to the deep dermis. They also tend to be more pronounced in wounds with a prolonged inflammatory phase and may develop in areas with increased mechanical tension. Both types of scars may produce considerable cosmetic disfigurement and prompt many patients affected to seek treatment. Although keloids can be successfully treated in a single application, patients may require multiple modes of therapy (Shaffer J J et al., J. Am. Acad. Dermatol. 2002; 46: 63-67).

Another type of scar tissue that may be treated by embodiments of the method and compositions of the present invention is Dupuytren's contracture. Dupuytren's contracture arises from unknown causes and is a progressive, scar-like shrinkage and thickening of the flexion contracture of the cusp-like extended palmar aponeurosis in the palm of the hand, whereby, as the curvature of the fingers increases, especially that of the fourth and fifth fingers, stretching of the fingers becomes ever more restricted. This ailment, which attacks men more frequently than women and can occur in one or both hands, begins with a dimple-like indentation in the palm of the hand and gradually but quite painlessly grows into nodules and fascicles. The flexor tendons of the fingers concerned are not in themselves diseased but their movement is impaired by the scar-fascicles of the palmar aponeurosis. A similar contracture concerning the toes is known.

Since the illness neither regresses spontaneously nor responds with any degree of long term success to conventional forms of treatment (without surgery) such as massage, heat treatment and the like, it can only be treated surgically, namely, by cutting away the proliferating atrophied tissue. In addition to ordinary risks and unpleasantness associated with any surgical operation, there exists a further risk that scars resulting from the operation can make a later recurrence of the ailment even worse.

One embodiment of the present invention is based, in part, on the discovery that the size and appearance of a closed wound or a scar can be improved, and the discomfort, itching, pain, and/or other symptoms caused by excessive tissue growth in a closed wound or scar can be alleviated (partially or completely) by the administration of at least one matrix metalloproteinase (MMP) modulator in combination with other pharmaceutically active agents selected out of (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents.

By “improving” the appearance of a closed wound or a scar according to an embodiment of the invention is meant alleviating, either partially or completely, symptoms such as pain, tingling, itching, burning, discoloration. Improving the appearance of a closed wound can include reducing the size of a closed wound or scar; reducing surface irregularities of the closed wound or scar; reducing the accumulation of fibrous tissue; and/or partially or completely eliminating the closed wound or the scar.

In one embodiment of the invention, a composition of the present invention is used to relieve or to prevent a condition of scar irritation. In particular, the compositions and methods of the present invention are useful in cases where scar irritation leads to symptoms including itching, and to a patient's self-inflicted mechanical action of scratching, which can result in further scar irritation, and possible contamination and invasion of the scar with native skin organisms.

The present invention may be comprised of a number of pharmaceutically active agents including one or more of matrix metalloproteinase modulators; cell cycle modulators; inflammatory event modulators, angiogenesis event modulators; fibroblast migration agents; fibroblast proliferation agents; tissue remodeling correcting agents; antimicrobial agents; modulators of deposition of extracellular matrix; drug penetration/permeation enhancer agents; antioxidative agents; antipuritic agents; fibrinolytic agents; immunomodulators; transcription modulating agents; surface modulating agents; growth factor inhibitors; and anti-proliferative agents.

One of ordinary skill in the art will appreciate that “matrix metalloproteinase modulator(s)” refers to any compound that modulates the activity of a matrix metalloproteinase. Thus, a MMP inhibitor or an MMP stimulator may be used in the present invention. Modulation of the activity of an MMP may result in a decrease in MMP activity, prohibition of MMP activity, stimulation of MMP activity, or an increase in MMP activity. Verapamil, integrins, antibodies to integrins, and pyramidine-2,4 diones are some examples of MMP modulators. One of ordinary skill in the art will appreciate that there are numerous other MMP modulators that may be used in practicing the present invention.

It will be understood that “cell cycle modulator(s)” refers to any compound modulates the cell cycle. Thus, a cell cycle inhibitor or a cell cycle stimulator may be used in the present invention. Modulation of the cell cycle may result in stimulating the cell cycle to progress or arresting progression of the cell cycle at any point during the cycle. Cyclins, such as cyclin B, cyclin dependent kinases, and stimulators of apoptosis, such as caspases, bad, and bax are some examples of cell cycle modulators. One of ordinary skill in the art will appreciate that there are numerous other cell cycle modulators that may be used in practicing the present invention.

One of ordinary skill in the art will appreciate that “inflammatory event modulator(s)” refers to any compound that modulates inflammation. Thus, in one example an anti-inflammatory may be used in the present invention. Modulation of inflammation may result in a decrease in inflammation, prohibition of inflammation, stimulation of inflammation, or an increase in inflammation. Cyclooxygenase inhibitors, including salicylic acid; acetylsalicylic acid; aryl, substituted or unsubstituted aralkyl, allyl, and substituted or unsubstituted, linear, branched, or cyclic alkyl esters of salicylic acid; aryl, substituted or unsubstituted aralkyl, allyl, and substituted or unsubstituted, linear, branched, or cyclic alkyl esters of acetylsalicylic acid; ibuprofen; celecoxib; rofecoxib; flufenamic acid; indomethacin; nabumetone; and naproxen, and NF-kB inhibitors, including salicylic acid; acetylsalicylic acid; aryl, substituted or unsubstituted aralkyl, allyl, and substituted or unsubstituted, linear, branched, or cyclic alkyl esters of salicylic acid; aryl, substituted or unsubstituted aralkyl, allyl, and substituted or unsubstituted, linear, branched, or cyclic alkyl esters of acetylsalicylic acid; nabumetone; sulindac sulfide; sulindac sulfone; sulfasalazine, and prostaglandin E2 inhibitors are some examples of inflammatory event modulators that may be used in the present invention. One of ordinary skill in the art will appreciate that there are numerous other inflammatory event modulators that may be used in practicing the present invention.

It will be understood that “angiogenesis event modulator(s)” refers to any compound modulates angiogenesis or events associated therewith. Thus, an angiogenesis event modulator may be a stimulator of angiogenesis or an inhibitor of angiogenesis. Modulation of events of angiogenesis may result in stimulating angiogenesis, arresting angiogenesis, increasing angiogenesis, decreasing angiogenesis, or preventing angiogenesis. Thalidomide, VEGF stimulators and inhibitors, bevacizumab, endostatin, onconase, and coramsine are some examples of angiogenesis event modulators. One of ordinary skill in the art will appreciate that there are numerous other angiogenesis event modulators that may be used in practicing the present invention.

One of ordinary skill in the art will appreciate that “fibroblast migration agent(s)” refers to any compound that modulates fibroblast migration. Thus, a fibroblast migration inhibitor or a fibroblast migration stimulator may be used in the present invention. Modulation of the migration of fibroblasts may result in a decrease or increase of fibroblasts. Seprase, dipeptidyl peptidase IV (DPPIV), tenacin-C, fibrinogen, TGF-beta 1, PKA, FGF, bFGF, beta-aminopropinitrile, prostaglandin E2, and ascorbate are some examples of fibroblast migration agents. One of ordinary skill in the art will appreciate that there are numerous other fibroblast migration agents that may be used in practicing the present invention.

It will be understood that “fibroblast proliferation agent(s)” refers to any compound causes fibroblasts to proliferate. Fibroblast growth factor is one example of a fibroblast proliferation agent. One of ordinary skill in the art will appreciate that there are numerous other fibroblast proliferation agents that may be used in practicing the present invention.

One of ordinary skill in the art will appreciate that “tissue remodeling correcting agent(s)” refers to any compound that corrects tissue remodeling. Correction of tissue remodeling may result in a decrease or increase of tissue remodeling. TNF-1, corticosteroids, and Fluticasone Propionate are some examples of tissue remodeling correcting agents. One of ordinary skill in the art will appreciate that there are numerous other tissue remodeling correcting agents that may be used in practicing the present invention.

It will be understood that “angiogenesis event modulator(s)” refers to any compound modulates angiogenesis or events associated therewith. Thus, an angiogenesis event modulator may be a stimulator of angiogenesis or an inhibitor of angiogenesis. Modulation of events of angiogenesis may result in stimulating angiogenesis, arresting angiogenesis, increasing angiogenesis, decreasing angiogenesis, or preventing angiogenesis. Thalidomide, VEGF stimulators and inhibitors, bevacizumab, endostatin, onconase, angiopoietin, fibroblast growth factor, transforming growth factor-beta and coramsine are some examples of angiogenesis event modulators. One of ordinary skill in the art will appreciate that there are numerous other angiogenesis event modulators that may be used in practicing the present invention.

It will be understood that “antimicrobial agent(s)” refers to any compound that kills, weakens, or prevents proliferation of microbials, such as E. coli bacteria. Some examples of antimicrobials that may be used in the present invention include aluminum hydroxide and aluminum zirconium trichlorohydrex. One of ordinary skill in the art will appreciate that there are numerous other antimicrobial agents that may be used in practicing the present invention.

One of ordinary skill in the art will appreciate that “modulator(s) of deposition of extra cellular matrix” refers to any compound that modulates deposition of the extra cellular matrix. Thus, such a compound may stimulate, increase, decrease or prevent deposition of the extracellular matrix. Laminins are some examples of modulators of deposition of extra cellular matrix. One of ordinary skill in the art will appreciate that there are numerous other modulators of deposition of extra cellular matrix that may be used in practicing the present invention.

It will be understood that “penetration enhancer” and “permeation enhancer” refer to any compound that enhance the penetration of compositions of the present invention and/or permeation of barriers such as the skin and cell walls. Some examples of penetration/permeation enhancers that may be used in the present invention include pyrrolidones, for example 2-pyrrolidone; alcohols, such as ethanol; alkanols, such as decanol; glycols, such as propylene glycol, dipropylene glycol, butylenes glycol; surfactants; glycerol derivatives, or terpenes. One of ordinary skill in the art will appreciate that there are numerous other penetration/permeation enhancers that may be used in practicing the present invention.

One of ordinary skill in the art will appreciate that “antioxidants” refers to naturally occurring or synthetic substances that inhibit or retard the oxidation of a substance to which it is added. They counteract the harmful and damaging effects of oxidation in animal tissues. Tocopherol, proanthocyanidins, and isoflavones are some examples of antioxidants. One of ordinary skill in the art will appreciate that there are numerous other antioxidants that may be used in practicing the present invention.

It will be understood that “antipuritic agent(s)” refers to any compound that prevents, lessens, or decreases itching or itching sensations. Some examples of antipuritic agents that may be used in the present invention include diphenhydramine, antihistamines, menthol, and camphor. One of ordinary skill in the art will appreciate that there are numerous other antipuritic agents that may be used in practicing the present invention.

One of ordinary skill in the art will appreciate that “fibrinolytic agent(s)” refers to fibrinolysin or agents that convert plaminogen to fibrinolysin to dissolve blood clots. Heparin, plasmin, plasminogen activator, protein C, protein S, and streptokinase are some examples of fibrinolytic agents. One of ordinary skill in the art will appreciate that there are numerous other fibrinolytic agents that may be used in practicing the present invention.

It will be understood that “immunomodulator(s)” refers to agents of both drug and biological origin often used in immunotherapy to stimulate, potentiate, or depress the immune response; also used to inhibit or enhance specific subclasses of immunocytes. Some examples of immunomodulators that may be used in the present invention include both immunosuppressants and immunostimulators. For example, interferons and interleukins are some examples of immunomodulators that may be used in the present invention. One of ordinary skill in the art will appreciate that there are numerous other immunomodulators that may be used in practicing the present invention.

One of ordinary skill in the art will appreciate that “transcription modulating agent(s)” refers to any compound that modulates transcription or any transcription factor. For example, transcription, or a transcription factor, may be stimulated, terminated, increased, or decreased. Polyamines, glucocorticoids, rapamyacin, actin, and protein kinase A are some examples of transcription modulating agents. One of ordinary skill in the art will appreciate that there are numerous other transcription modulating agents that may be used in practicing the present invention.

It will be understood that “surface modulating agent(s)” refers to agents of both drug and biological origin used in modulating a surface, for example the surface of a closed wound. The surface modulating agent may increase the surface, decrease the surface, improve the surface, irritate the surface or have some other effect on the surface. Integrins are an example of a class of surface modulating agents. One of ordinary skill in the art will appreciate that there are numerous other surface modulating agents that may be used in practicing the present invention.

One of ordinary skill in the art will appreciate that “growth factor inhibitor(s)” refers to any compound that inhibits growth factors expression, stimulation or activity. Inhibitors of fibroblast growth factor, vascular endothelial growth factor, epidermal growth factor, and transforming growth factor are some examples of growth factor inhibitors. One of ordinary skill in the art will appreciate that there are numerous other growth factor inhibitors that may be used in practicing the present invention.

It will be understood that “antiproliferative agent(s)” refers to agents of that prevent proliferation, for example cellular proliferation. Some examples of antiproliferative agents that may be used in the present invention include paclitaxel, leflunomide, and didemnin B. One of ordinary skill in the art will appreciate that there are numerous other antiproliferative agents that may be used in practicing the present invention.

Administration

To achieve the improvements described herein, the present invention provides for administering a composition comprised of a hydrophilic or hydrophobic carrier (or a mixture of the same), wherein the composition comprises at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents. For example, the composition to be administered may comprise verapamil hydrochloride, an MMP modulator, in combination with Vitamin E, Aloe Vera and/or Salicylic acid. A composition of the present invention may be orally, topically, or transdermally administered, administered by injection, administered by inhalation, or administered by a combination of two or more of these administration routes.

For topical or transdermal administration, one embodiment of the invention encompasses placing the composition comprising at least one matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents directly on the surface of the scar or closed wound.

The compositions of the present invention are not intended for use on infected skin or on open wounds.

In another embodiment, a composition for use in improving the appearance and reducing the size of a closed wound or scar is placed in contact with the affected area of skin; the composition may then covered with a thermal insulating material or a PEG material. In each embodiment described herein, the composition placed in contact with the affected area of skin is allowed to remain in place for a period of time sufficient to bring about an improvement in the appearance and/or a reduction in the size of the closed wound or scar.

Although there is no minimum time required for duration of use, the duration of use of a composition of the present invention can extend for example, from about 0.5 hour to about 24 hours, or from about 1 hour to about 1 month, or from about 8 hours to about 12 months or from about 24 hours to about 24 months. In one embodiment of the invention, the duration of use typically extends from about 12 hours to about 12 months, for separate time periods. The composition can be used continuously or intermittently for a particular time period and then removed or reapplied. For example, the composition can be used intermittently from about 1 hour to about 72 hours, from about 8 hours to about 48 hours, from about 12 hours to about 24 hours, or from about 18 hours to about 24 hours. The time interval between each use can be from about 1 hour to about 72 hours, from about 8 hours to about 48 hours, from about 12 hours to about 24 hours, or from about 18 hours to about 24 hours. In a particular embodiment, each time period is about 18 hours in a day with a minimum of about 4 hours between time periods.

In one embodiment in which topical administration is used to administer a composition according to an embodiment of the invention, the use of adhesive tape is avoided because adhesives may cause irritation of a scar and aggravate the scar condition. It is recommended that means such as flexible wraps, elastic garments, netting, ace wraps or spandex sleeves or garments be used to affix a composition according to the invention to the affected area of skin.

In one embodiment, a composition of the present invention comprising at least at least one matrix metalloproteinase (MMP) modulator in combination with a PEG material and one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents is topically administered. In particular, a layer of the composition is topically applied over the closed wound or scar in a range of about 0.0625″-0.125″ in depth of composition and in a range of 1-3 times daily over an interval of time that results in an improvement of the closed wound or scar.

In addition to compositions suitable for topical or transdermal administration to the affected area of skin, in another embodiment of the invention, compositions may be those suitable for oral or parenteral (including intramuscular, sub-cutaneous and intravenous) administration, or those in a form suitable for administration by inhalation. The therapeutically effective substance of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use; or in the form of sterile injectable solutions for parenteral (including sub-cutaneous) use.

In one embodiment of the invention, aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like. Solid form preparations include, among others, powders, tablets, pills, capsules, and dispersible granules. A solid carrier can be one or more substances which may also act as diluents, flavoring agents, solubilizers, lubricants, suspending agents, binders, preservatives, tablet disintegrating agents, or an encapsulating material. In powders, the carrier is a finely divided solid which is in a mixture with the finely divided active component. In tablets, the active component is mixed with the carrier having the necessary binding capacity in suitable proportions and compacted in the shape and size desired.

According to an embodiment of the invention, powders and tablets preferably contain from five or ten to about seventy percent of the active compound. Suitable carriers are magnesium carbonate, magnesium stearate, sugar, lactose, pectin, dextrin, starch, gelatin, tragacanth, methylcellulose, sodium carboxymethylcellulose, a low melting wax, cocoa buffer, and the like. The term “preparation” is intended to include the formulation of the active compound with encapsulating material as carrier providing a capsule in which the active component, with or without carriers, is surrounded by a carrier, which is thus in association with it. Similarly lozenges are included. Tablets, powders, capsules, pills and lozenges can be used as solid forms suitable for oral administration.

According to an embodiment of the invention, liquid preparations include solutions, suspensions, and emulsions, for example, sterile water or water-propylene glycol solutions. For example, parenteral injection liquid preparations can be formulated with PEG in aqueous solution. Other suitable pharmaceutical carriers for parenteral administration include, for example, physiological saline, bacteriostatic saline (saline containing about 0.9% mg/ml benzyl alcohol), phosphate-buffered saline, Hank's solution, Ringer's-lactate and the like. An embodiment of a therapeutically effective substance according to the present invention may thus be formulated for parenteral administration (by injection, for example, by bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

According to an embodiment of the invention, administration may also be made to the respiratory tract by means of an aerosol formulation in which the active ingredient is provided in a pressurized pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dosage of the therapeutic substance may be controlled by provision of a metered valve. In compositions intended for administration to the respiratory tract, including intranasal compositions, compounds used in an embodiment will generally have a small particle size, for example of the order of 5 microns or less. Such a particle size may be obtained by means known in the art, for example by micronization.

While a composition for use in improving the appearance and/or reducing the size of a closed wound or scar may be administered in the form of a raw chemical compound, including a physiologically acceptable salt of the active ingredient, it is preferred to introduce the active ingredient, e.g. the MMP in combination with one of the above-identified pharmaceutically active agents, in a pharmaceutical composition together with a pharmaceutically acceptable carrier and one or more adjuvants, excipients, and/or diluents.

In addition, the compositions of the present invention may further comprise an anti-irritant substance, a deodorant agent, such as aluminum hydroxide, an anti-microbial substance such as aluminum zirconium trichlorohydrex, or other metallic anti-microbial. Additionally, the composition may further comprise penetration enhancers, buffers, preservatives, emulsifiers, or emollients.

One of ordinary skill in the art will appreciate that any of the compositions of the present invention may be administered with a suitable pharmaceutical carrier that may be hydrophilic, hydrophobic or some combination of hydrophilic and hydrophobic. The choice of the pharmaceutical carrier depends on the route of administration and the size of the scar. The terms “suitable pharmaceutical carrier” and “pharmaceutically acceptable carrier” and grammatical variations thereof, as they refer to compositions, carriers, diluents and reagents, are used herein interchangeably. As the terms are used herein, “suitable pharmaceutical carrier” and “pharmaceutically acceptable carrier” refer to non-toxic materials that do not interfere with the effectiveness of the biological activity of active ingredients, and represent that the materials are capable of administration to or upon a vertebrate with a minimum of undesirable physiological effects such as nausea, dizziness, gastric upset and the like. The characteristics of the pharmaceutically acceptable carrier will depend on the route of administration.

The preparation of a pharmacological composition that contains an MMP modulator in combination with one of the above-identified pharmaceutical agents, i.e., the active ingredients, dissolved or dispersed therein is well understood in the art and need not be limited based on formulation. Liquid preparations include solutions, suspensions, colloids, hydrogels, PEGs, and emulsions, for example, water, water-propylene glycol mixtures. Such compositions may be prepared as injectables, either as liquid solutions or suspensions, or as topical applications, for example, as salves, lotions, creams. Alternatively, compositions of the present invention can be administered as an aerosol product to spray-on a closed wound.

Solid forms of such a pharmacological composition suitable for dissolving in a hydrogel, a PEG material, or a liquid solution, or for suspending in liquid prior to use, can also be prepared. The preparation can also be emulsified. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. Details on techniques for formulation and administration may be found in the latest edition of Remington's Pharmaceutical Sciences (Maack Publishing Co., Easton, Pa.).

As used herein, the term “thermal insulating material” includes materials that, when placed in contact with or near to the skin, are capable of retaining sufficient heat to elevate the surface temperature of the affected area of the skin.

In one embodiment of the invention, the thermal insulating material when used to cover the affected area causes an elevation in the surface temperature of the closed wound or scar of from about 0.5° C. to about 5° C. In another embodiment, the thermal insulating material, when used to cover the affected area, causes an elevation in the surface temperature of the closed wound or scar of from about 1° C. to about 4° C. In a preferred embodiment, the thermal insulating material, when used to cover the affected area, causes an elevation in the surface temperature of the closed wound or scar of from about 2° C. to about 3° C.

In one embodiment, the thermal insulating material may be a sponge. Examples of sponge materials suitable for use as a thermal insulating material in the present invention include collagen and cross-linked collagen. The term “cross-linked,” as used herein, refers to covalent bonds formed among polymeric chains and to an interconnected structure wherein cross-links are formed between hydrophobic molecules, between hydrophilic molecules and between hydrophobic molecules and hydrophilic molecules.

In another embodiment, the thermal insulating material may be a gel, a hydrogel, or a biodegradable hydrogel. Gels and hydrogels generally contain a very high concentration of water, e.g., about 60% to about 98% water and are held together by a variety of cellular groups. The water may be bound in the form of various hydrates, or unbound, entrapped in cellular pockets formed by the polymer network groups.

The term “hydrogel” is used herein to mean a polymeric material which can include a cross-linked macromolecular network, which exhibits the ability to swell in water and to retain a significant portion of water within its structure without dissolving.

A “biodegradable hydrogel,” as the term is used herein, is a hydrogel formed from a hydrogel-forming system containing at least one biodegradable component, i.e., a component which is degraded by water and/or by enzymes found in nature.

There are a number of well-known hydrophilic, polymeric compounds both naturally occurring and synthetic, which form networks, creating a gel in the presence of water. For example, gelatin can be obtained from the hydrolysis of collagen by boiling skin, ligaments, tendons, etc. A mixture of only 2% gelatin in water will form a stiff gel. An example of a gel suitable for use in an embodiment of the invention is Elastogel®, available from Southwest Technologies, Kansas City, Mo.

A hydrogel may be formed by adding a solute such as gelatin to water at an elevated temperature to dissolve gelatin. The solution is then cooled and the solute(s) (e.g., solid gelatin components) form submicroscopic crystalline particle groups which retain a great deal of water in the interstices (so-called “brush-heap” structure). Methods of making hydrogels suitable for use in the present invention are well-known to those of skill in the art. See, for example, the disclosures of U.S. Pat. No. 4,646,730 to Schonfeld et al.; U.S. Pat. No. 5,013,769 to Murray et al.; U.S. Pat. No. 4,659,700 to Jackson et al.; and U.S. Pat. No. 4,909,244 to Quarfoot et al., the teachings of which are incorporated herein by reference in their entireties. An example of a hydrogel suitable for use in an embodiment of the invention is AVOGEL®, available from Avocet Polymer Technologies, Inc., Chicago, Ill.

In addition to increasing the surface temperature of the closed wound, the thermal insulating material may also be used to deliver a therapeutically effective substance to the closed wound. Alternatively, a PEG material may be used to deliver a therapeutically effective substance to the closed wound. As used herein, the terms “therapeutically effective substance” or “therapeutic substance” include:

(i) Compounds and compositions recognized in the official United States Pharmacopoeia, the official Homeopathic Pharmacopoeia of the United States, or the official National Formulary, or any supplement of any of them;

(ii) Compounds and compositions intended for use in the diagnosis, cure, mitigation, treatment, or prevention of disease in man or other animals; and

(iii) Compounds and compositions (other than food) intended to affect the structure or any function of the body of man or other animals.

As used herein, a “PEG material” refers to a polyethylene glycol (PEG) or a blend of at least two PEGs that are of differing molecular weights. Polyethylene glycols are water-soluble, odorless, neutral, lubricating, nonvolatile and nonirritating liquid compositions. Polyethylene glycols are composed of linear polymers formed by the addition reaction of ethylene oxide. The generalized formula for PEG is H—(OCH₂CH₂)ₙ—OH where “n” is the average number of repeating oxyethylene groups. A PEG is typically designated by a number that represents its average molecular weight. For example, a PEG 600 consists of a distribution of polymers of varying molecular weights with an average of 600, which corresponds to an approximate average number of repeating oxyethylene groups (“n”).

It is the repeating ether linkages and terminal hydroxyl groups of PEGs that gives rise to the water solubility of PEGs. Polyethylene glycols also are soluble in organic solvents such as acetone, alcohols, and chlorinated solvents, while they are insoluble in nonpolar solvents such as hydrocarbons.

Polyethylene glycols are hygroscopic, i.e., they attract and retain moisture from the atmosphere. This property makes PEGs useful as water-soluble ointments and humectants, and as replacements for other hygroscopic materials such as glycerin and propylene glycol in certain applications. Hygroscopicity decreases as the molecular weight of the PEG increases.

Above their melting/freezing temperatures, PEGs can be considered Newtonian fluids since their viscosities are nearly independent of shear. Kinematic viscosity measurements, therefore, are the most practical way of characterizing PEG viscosity. Viscosities of PEG materials decrease as temperature increases.

Polyethylene glycols may be liquids or solids at room temperature, depending on the average molecular weight of the PEG. Higher molecular weight PEGs form more rigid solids, while lower molecular weight PEGS tend to be viscous liquids at room temperature. For example, PEGs having average molecular weights from 200-600 are typically clear, viscous liquids at room temperature; PEGs having average molecular weights from 900-1500 are typically soft, opaque white solids at room temperatures; and PEGs having average molecular weights from 3350-8000 typically are hard, opaque white solids at room temperature. Increased molecular weight results in decreased solubility in water and other solvents, decreased hygroscopicity and vapor pressure, and increased melting/freezing range and viscosity. Intermediate physical properties can be achieved by blending PEGs of differing molecular weights. For example, blending two PEGs, for example a 400 MW PEG and a 3350 MW PEG can lead to a composition with the consistency of an ointment or salve, or a composition having a gel tendency.

Polyethylene glycols make excellent water-soluble ointment bases; they spread easily and evenly over the skin, even if the skin is moist. The good water solubility of PEGs makes it easy to incorporate aqueous ingredients in the formulation, and they do not become rancid or support microbial growth.

Additionally, in practicing the present invention, the PEG material may create a waxy gel on the surface of the closed wound or scar that decreases UV light penetration and buffers superoxide radicals. PEG materials preferably limit the area of treatment to an appropriate level of moisture. Further, the structure of the PEG material provides a mechanical barrier for decreased penetration of bacteria and foreign materials. The PEG material also creates an evaporative barrier which augments transdermal delivery of the drugs into the closed wound or scar area. In one embodiment, the PEG material comprises an acidic composition, which is favorable for the skin and for longevity of the PEG material.

In one embodiment, one or more therapeutically effective substances may be applied to one surface of a thermal insulating material. The thermal insulating material is then applied to the closed wound in a manner such that the therapeutically effective substance is placed in contact with the closed wound.

In another embodiment, the therapeutically effective substance is dispersed within a hydrogel, a water-insoluble gel, a sponge or a PEG material. The hydrogel, water-insoluble gel, sponge or PEG material within which the therapeutically effective substance is dispersed, is then placed in contact with the affected surface of the skin, and allowed to remain in place for a period of time sufficient to bring about an improvement in the size and appearance of the closed wound.

As used herein, the term “dispersed” includes ionic, covalent, hydrophilic, or hydrophobic interactions between the therapeutically effective substance and the hydrogel, water-insoluble gel, sponge, or PEG material.

For example, a therapeutically effective substance containing a cationic moiety can be immobilized on a hydrogel polymer chain. As will be recognized by those skilled in the art, this cationic site may serve as a noncovalent, ionic binding site for anionic substances, such as certain NSAIDs.

In another example, a hydrogel or sponge can be chosen which covalently bonds to the therapeutic substance used according to one embodiment. For example, through hydrophilic interactions with water in the hydrogel, any water soluble drug will dissolve in the hydrogel. A hydrophobic interaction between a non-water soluble therapeutic substance and a hydrogel can occur when the hydrogel selected includes a hydrophobic entity which is receptive to further interaction with a therapeutic substance having a hydrophobic moiety.

One skilled in the art will know, or will be able to ascertain with no more than routine experimentation, what hydrogels or PEG materials are suitable for dispersing a particular therapeutic substance.

A therapeutic substance which covalently bonds to the hydrogel, sponge or PEG material can form a drug delivery substance with controlled or sustained release. If a biodegradable hydrogel or sponge is used, delivery of the therapeutic substance to the closed wound or scar is also related to the rate of degradation of the hydrogel or sponge. The degradation rate of the hydrogel or sponge is usually slower than the diffusion rate of the therapeutic substance. As is well-known to those of skill in the art, by choosing a particular concentration of each therapeutic substance used in a particular embodiment, and a particular hydrogel or sponge, one can control the rate of degradation or the rate of diffusion, and thus, the rate of delivery of the therapeutic substance.

The hydrogel, other thermal insulating material, or PEG material containing the therapeutically effective substance can remain in contact with the surface of the affected area of skin for about between 0.5 to about one hour per day, from about one hour to about 8 hours per day, from about 12 hours to about 15 hours per day, from about 12 hours to about 18 hours per day, from about 18 hours to about 24 hours per day, or over a number of days, for a sufficient number of days to bring about an improvement in the size and appearance of the closed wound or scar. The hydrogel, other thermal insulating material, or PEG material can be removed periodically in order to cleanse the scar surface and to apply a fresh sample of therapeutically effective substance and hydrogel, other thermal insulating material, or PEG material.

In one embodiment, a composition of the present invention is administered topically with a suitable pharmaceutical carrier, including one or more substances that relieve skin irritation. In a particular embodiment of the invention wherein the method of administration is topical, the substance that relieves skin irritation includes at least one of the following substances: glyceryl monooleate, diphenhydramine, calamine, and a C₃-C₄ diol.

In one embodiment, a closed wound, such as a scar, is contacted with a hydrogel or PEG material comprising at least one a cyclooxygenase inhibitor, for example, ibuprofen, indomethacin, sodium salicylate, or at least one NF-kB inhibitor, for example, acetyl salicylic acid, sulfasalazine, or combinations thereof, and a deodorant agent to reduce surface bacteria and odor formation.

In one embodiment, a closed wound is treated by contacting the closed wound with a hydrogel or a PEG material comprising an effective amount of salicylic or derivatives/analogues thereof in a pharmaceutically acceptable carrier. The hydrogel preferably elevates the surface temperature of the affected area of skin. The hydrogel or PEG material is allowed to remain in contact with the affected area of skin for a period of time sufficient to result in an improvement in the closed wound.

Examples of suitable patterns of use according to an embodiment of the invention include, among others: use of various hydrogel or PEG material combinations in sequence; use of various hydrogel or PEG material combinations simultaneously; use of various hydrogel or PEG material combinations in systemic-topical co-administration, such as oral administration simultaneously with topical administration; use of combinations of active ingredients mixed by a pharmacist according to a prescription; and use of combinations of separate active ingredients available in kit form, mixed by the patient and self-administered according to physician instructions or directions provided with the kit.

Therapeutically Effective Amount and Dosage

As used herein, the terms “therapeutically effective amount” and “therapeutically effective dose” refer to the amount of an active agent, for example, a composition comprising matrix metalloproteinase (MMP) modulator in combination with one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; (p) anti-proliferative agents, or an anti-irritant, required to be administered in order to induce a desired result in the patient. That result may be alleviation or amelioration (complete or partial) of the symptoms or condition of irritation, pain, tingling, redness or other discoloration of a closed wound, an improvement in the appearance or reduction in the size of the closed wound, or any other desired improvement in the affected area of skin.

As used herein, the term “therapeutically effective amount” may also refer to the quantity of active agent or therapeutically effective substance, the administration of which results in improvement in the size, appearance, or condition of a closed wound, where little or no improvement would occur in the absence of the active agent. Typically, the active agent is administered for a sufficient period of time to achieve the desired therapeutic effect.

Therapeutic efficacy may be determined as described herein and by using standard pharmacological procedures in experimental animals.

The active ingredient of an embodiment of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous) use, or in the form of aerosol formulations for inhalation therapy. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Further details on techniques for formulation and administration may be found in the latest edition of Remington's Pharmaceutical Sciences (Merck Publishing Co., Easton, Pa.). When desired, compositions adapted to give sustained release of the active ingredient may be employed.

The dose administered is adjusted to the size and severity of the closed wound or affected area of skin, the age, weight and condition of the individual being treated, as well as the route of administration, dosage form and regimen, and the result desired. The exact dosage should of course be determined by the practitioner.

The active ingredient can be administered in one or several doses per day. In one embodiment, it is presently contemplated that, for therapeutic treatments, at least one composition of the present invention, such as a MMP modulator in combination with a cyclooxygenase inhibitor or an anti-irritant, can be administered in an amount comprising from about 1 microgram to about 3000 micrograms, from about 10 micrograms to about 2000 micrograms, from about 20 micrograms to about 1000 micrograms, or from about 40 micrograms to about 400 micrograms per square centimeter of treated tissue.

In another embodiment, for therapeutic treatments, at least one MMP modulator is administered in an amount comprising from about 1 microgram to about 2000 micrograms, from about 10 micrograms to about 1000 micrograms, or from about 40 micrograms to about 400 micrograms per square centimeter of treated tissue. The amount of composition of the present invention can be administered by any suitable method of administration, including, but not limited to, topical application, subcutaneous or parenteral administration, oral administration, administration by inhalation, and by combinations of these methods.

In one embodiment, the amount of composition of the present invention can be included in an amount from about 1 percent by weight to about 75 percent by weight, from about 5 percent to about 50 percent by weight, or from about 10 percent to about 40 percent by weight, in a thermal insulating material or a PEG material. In a particular embodiment, the composition of the present invention is included in an amount of about 40 percent by weight in a thermal insulating material or PEG material.

In another embodiment of the present invention, the PEG material of the present invention comprises a first PEG of 200-600 MW blended with a second PEG of 3350-5000 MW as a pharmaceutical carrier and anti-bacterial adhesion agent. For example, the first PEG may have a molecular weight of 400 and may form 45-75% of the pharmaceutical carrier while the second PEG may have a molecular weight of 3350 and may form 25-55% of the pharmaceutical carrier.

In a further embodiment, the PEG material of the present invention further comprises methyl paraben, propyl paraben, aloe vera, vitamin E, and salicylic acid. For example, methyl paraben may form approximately 0.001-1% of the composition, propyl paraben may form 0.001-1%, aloe vera may form from approximately 0.001-2%, vitamin E may form from approximately 0.001-2%, and verapamil may form from 0.1-10% of the composition.

In a further example, a PEG material of the present invention comprises: a first PEG having a molecular weight of 200-600, wherein the first PEG forms 45-75% of the composition; a second PEG having a molecular weight of 3350-500, wherein the second PEG forms 25-55% of the composition; methyl paraben, wherein the methyl paraben forms approximately 0.2% of the composition; propyl paraben, wherein the propyl paraben forms approximately 0.1% of the composition; aloe vera, wherein the aloe vera comprises approximately 0.001% of the composition; vitamin E, wherein the vitamin E forms approximately 0.001% of the composition; and at least one matrix metalloproteinase (MMP) modulator in combination with a PEG material and one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents. The composition can be administered topically to deliver an amount comprising from about between about 1 microgram to about 3000 micrograms, from about 10 micrograms to about 2000 micrograms, from about 20 micrograms to about 1000 micrograms, or from about 40 micrograms to about 400 micrograms, for example, of an MMP modulator in combination with an NF-kB inhibitor or cyclooxygenase inhibitor, per square centimeter of treated tissue.

For example, in one embodiment, a closed wound is treated by contacting the closed wound with a hydrogel or a PEG material comprising an matrix metalloproteinase (MMP) modulator in combination with a PEG material and -one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents present in an amount up to about 40 percent of the weight of the hydrogel or PEG material; the amount of the composition of the present invention that is administered comprises from about 1 microgram to about 2000 micrograms, or from about 40 micrograms to about 400 micrograms of composition of the present invention per square centimeter of treated tissue. Wherein the closed wound is treated using a hydrogel according to the present invention, the surface temperature of the closed wound is elevated by the hydrogel from about 0.5° C. to about 5° C.

In another embodiment, a closed wound is treated by contacting the closed wound with a thermal insulating material or a PEG material including an effective amount of at least one anti-irritant compound in a suitable pharmaceutical carrier. Examples of an anti-irritant compound or substance that relieves skin irritation that can be used according to this embodiment of the invention include: glyceryl monooleate, diphenhydramine, calamine, and a C₃-C₄ diol. The amount of anti-irritant compound used in this embodiment comprises from about 1 microgram to about 2000 micrograms, or from about 40 micrograms to about 400 micrograms of anti-irritant compound per square centimeter of treated tissue. The thermal insulating material elevates the surface temperature of the closed wound. The thermal insulating material or the PEG material is allowed to remain in contact with the affected area of skin.

The amount of the composition of the present invention that is administered, and the dosing regimen used, will, of course, be dependent on the particular drug selected, the route or routes of administration employed, the age and general condition of the subject being treated, the severity of the subject's condition, and the judgment of the prescribing physician. Generally, the daily dosage when administered topically or by injection will be determined by, among other factors, the dosage which may be given by some other mode of administration, such as oral. Alternatively, a large initial loading dose can be used to achieve effective levels of the agent, and can be followed by smaller doses to maintain those levels.

In another embodiment, the invention comprises a kit, or packaged assembly, in the form of a consumer package or prescription package which provides the necessary ingredients described herein together with directions on how to combine the ingredients to make a hydrogel for use in treatment of a closed wound (e.g., to treat a condition of scar irritation, or to reduce hypertrophic scarring). In one embodiment, a kit may include ingredients that can be co-administered with a hydrogel or PEG material by mixing one or more ingredients with the hydrogel or PEG material for topical application, or for another mode of administration such as oral.

In one embodiment, a kit for improving the appearance and/or reducing the size of a closed wound can include a composition of the present invention, matrix metalloproteinase (MMP) modulator in combination with a PEG material and one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents or an anti-irritant and a hydrogel or a PEG material. Such a kit may further include a sterile solution (e.g., saline, water) for mixing with the composition of the present invention. The composition can be applied to the closed wound and covered with the hydrogel or PEG material. In another embodiment, a kit may include a hydrogel or PEG material that includes a composition of the present invention such as a MMP modulator in combination with a cyclooxygenase inhibitor or an NF-kB inhibitor.

In another embodiment, a kit may include a hydrogel or a PEG material and a composition comprising up to about 35 percent of each of the following: salicylic acid or a derivative thereof; acetylsalicylic acid or a derivative thereof; a compound selected from aluminum hydroxide, aluminum zirconium trichlorohydrex, and other metallic anti-microbials; a compound selected from diphenhydramine and other anti-pruritic agents; matrix metalloproteinase (MMP) modulator in combination with a PEG material and one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents.

In a further embodiment, the kit comprises a PEG material comprising a PEG of 400 MW combined with a PEG of 3350 MW as a pharmaceutically acceptable carrier. For example, the PEG of 400 MW may form 45-75% of the pharmaceutical carrier while the PEG of 3350 MW may form 25-55% of the pharmaceutical carrier. The PEG material included in the kit of the present invention may further comprise methyl paraben, propyl paraben, aloe vera, vitamin E, an MMP, and salicylic acid or a derivative thereof. For example, methyl paraben may form approximately 0.001-1% of the composition, propyl paraben may form 0.001-1%, aloe vera may form from approximately 0.001-2%, vitamin E may form from approximately 0.001-2% and salicylic acid, or a derivative/analogue thereof, may form from about 1-5% of the composition.

In another embodiment the kit may include, in addition to active ingredients and other materials for topical administration, a cyclooxygenase inhibitor and/or an anti-irritant such as diphenhydramine for oral administration.

In one embodiment, a kit may include a hydrogel composition including up to 20% by weight PEG with up to 95% by weight sterile water. Because PEG is most efficiently sterilized when in powder form, the preparation of the hydrogel by the consumer or patient increases the quality of available gels and reduces the cost of the scar control therapy by leaving the addition of water to the PEG powder until immediately prior to use.

In addition, a kit-based preparation of a hydrogel or a PEG material which includes, in one embodiment, an anti-pruritic ingredient and/or matrix metalloproteinase (MMP) modulator in combination with a PEG material and one or more of the following pharmaceutically active agents: (a) cell cycle modulators; (b) inflammatory event modulators; (c) angiogenesis event modulators; (d) fibroblast migration agents; (e) fibroblast proliferation agents; (f) tissue remodeling correcting agents; (g) antimicrobial agents; (h)modulators of deposition of extra cellular matrix; (i) drug penetration/permeation enhancer agents; (j) antioxidative agents; (k) antipuritic agents; (l) fibrinolytic agents; (j) immunomodulators; (m) transcription modulating agents; (n) surface modulating agents; (o) growth factor inhibitors; or (p) anti-proliferative agents, and/or a topical antimicrobial, permits a consumer or patient access to a therapy individually tailored or designed to the size and severity of the irritated scar condition. The components can be prescribed by a treating clinician or can be self-selected based on the patient's current assessment of the scar condition.

A closed wound control kit according to an embodiment can include devices such as “control top” panties or other garments for use with scars of the lower abdominal skin, stretch bandages (e.g. elastic-type sports wraps or ace wraps), and gloves to keep the hydrogel or the PEG material affixed to the affected area of skin.

Materials in a kit may also include: protective gloves for user to wear during hydrogel or PEG material preparation; miniature timer to allow user to time the hydrogel or PEG material preparation; miniature spatula to smooth or stir hydrogel or PEG material ingredients; hydrogel or PEG material “tray” or “mold” of depth not to exceed 0.5 cm, and length and width varying depending on the amount of hydrogel or PEG material to be prepared by user; a ruler either in paper, plastic, or tape form, to allow user to measure affected skin to be treated and to choose correctly the size and amount of hydrogel or PEG material to be prepared.

An example of a method according to one embodiment of using a kit may include reading directions prior to kit use; using included ruler to measure affected area of skin; comparing scar size to hydrogel or PEG material preparation table for ingredients and recipe or to prescription or clinician recommendation; using spatula to mix recommended amount of water with hydrogel or PEG material ingredients in amount specific to scar size; placing hydrogel or PEG material mixture in mold or tray and timing; adding active ingredients according to prescription or as recommended by clinician or by personal preference; applying resulting hydrogel or PEG material to affected area of skin and securing the hydrogel or PEG material to the skin. A method according to an embodiment may also include co-administering other compositions depending on prescription, as recommended by clinician, or according to personal preference.

A kit according to an embodiment may include ingredients or components for one treatment or may include ingredients or components for multiple treatments. A kit according to an embodiment may include a combination of medications and devices tailored to a patient's irritated scar condition.

It is intended that the foregoing detailed description be regarded as illustrative rather than limiting. The present invention is further illustrated by the following experimental investigations and examples, which should not be construed as limiting. The contents of all references, patents and published applications cited throughout this patent are hereby incorporated by reference herein.

### EXAMPLE 1

### Manufacture of PEG Material including the MMP modulator Verapamil

In one example, the PEG material of the present invention may be manufactured by combining approximately 718 pounds of PEG 400 (e.g. Carbowax® available from The Dow Chemical Company®) with approximately 308 pounds of PEG 3350 (e.g. Carbowax® available from The Dow Chemical Company®) in a large stainless steel kettle. Preferably, the kettle has been cleaned and sanitized prior to combining the PEG materials. The combined PEG materials are heated until the temperature reaches 65° C. The combined PEG materials are mixed until they are completely in liquid form.

Once the combined PEG materials are liquid, the liquid PEG is maintained at 65° C. and methyl parabenzene, propyl parabenzene, aloe vera powder, vitamin E, and salicylic acid are added. Then the mixture is cooled to 40° C. At room temperature, approximately 25° C., 10% w/w verapamil hydrochloride is added by stirring.

Listed below in Table 1 is a formulation that may be used to produce a composition of the present invention according to the methods described in this example.

TABLE 1
Formulation

| | |
|---|---|
| Verapamil | 10% |
| PEG 400 | 61.55% |
| PEG 3350 | 23.69 |
| Salicylic Acid | 1.8% |
| Vitamnin E acetate | 0.001% |
| Parabens | 0.27% |
| Aloe Vera | 0.001% |

### EXAMPLE 2

### Alternative Formulation of PEG including Verapamil

In another example, the PEG material of the present invention may be manufactured by combining approximately 718 pounds of PEG 400 (e.g. Carbowax® available from The Dow Chemical Company®) with approximately 308 pounds of PEG 3350 (e.g. Carbowax® available from The Dow Chemical Company®) in a large stainless steel kettle. Preferably, the kettle has been cleaned and sanitized prior to combining the PEG materials. The combined PEG materials are heated until the temperature reaches 65° C. The combined PEG materials are mixed until they are completely in liquid form.

Once the combined PEG materials are liquid, the liquid PEG is maintained at 65° C. and methyl parabenzene, propyl parabenzene, aloe vera powder, and vitamin E acetate are added.

Verapamil HCl is dissolved into ethanol and added to the liquid PEG solution at a temperature of approximately 60-70° C. The mixture is allowed to cool to a temperature of approximately 31-35° C.

Listed below in Table 2 is a formulation that may be used to produce a composition of the present invention according to the methods described in this example.

TABLE 2
Alternative Formulation

| | |
|---|---|
| Verapamil | 6% |
| PEG 400 | 49.39% |
| PEG 3350 | 21.19% |
| Ethanol | 23% |
| Vitamnin E acetate | 0.001% |
| Parabens | 0.25% |
| Aloe Vera | 0.001 |

### EXAMPLE 3

### Further Alternative Formulation of PEG including Verapamil

In another example, the PEG material of the present invention may be manufactured by combining approximately 718 pounds of PEG 400 (e.g. Carbowax® available from The Dow Chemical Company®) with approximately 308 pounds of PEG 3350 (e.g. Carbowax® available from The Dow Chemical Company®) in a large stainless steel kettle. Preferably, the kettle has been cleaned and sanitized prior to combining the PEG materials. The combined PEG materials are heated until the temperature reaches 65° C. The combined PEG materials are mixed until they are completely in liquid form.

Once the combined PEG materials are liquid, the liquid PEG is maintained at 65° C. and methyl parabenzene, propyl parabenzene, aloe vera powder, and vitamin E acetate are added.

Verapamil HCl is directly dissolved in the liquid PEG solution containing methyl parabenzene, propyl parabenzene, aloe vera powder, and vitamin E acetate at a temperature of approximately 60-70° C. The mixture is allowed to cool to a temperature of approximately 31-35° C.

Listed below in Table 3 is a formulation that may be used to produce a composition of the present invention according to the methods described in this example.

TABLE 3
Further Alternative Formulation

| | |
|---|---|
| Verapamil | 6% |
| PEG 400 | 59.03% |
| PEG 3350 | 34.6% |
| Vitamnin E acetate | 0.001% |
| Parabens | 0.25% |
| Aloe Vera | 0.001% |

1. A composition for reducing the size or improving the appearance of a closed wound comprising a hydrophilic carrier or a hydrophobic carrier or a combination thereof, wherein the composition further comprises at least one matrix metalloproteinase (MMP) modulator in combination with at least one pharmaceutically active agent selected from the group consisting of:
(a) cell cycle modulators;
(b) inflammatory event modulators;
(c) angiogenesis event modulators;
(d) fibroblast migration agents;
(e) fibroblast proliferation agents;
(f) tissue remodeling correcting agents;
(g) antimicrobial agents;
(h) modulators of deposition of extra cellular matrix;
(i) penetration enhancers;
(j) antioxidants;
(k) antipuritic agents;
(l) fibrinolytic agents;
(j) immunomodulators;
(m) transcription modulating agents;
(n) surface modulating agents;
(o) growth factor inhibitors; and
(p) antiproliferative agents.

2. A medical device for reducing the size or improving the appearance of a closed wound comprising a hydrophilic carrier or a hydrophobic carrier or a combination thereof, wherein the composition further comprises at least one matrix metalloproteinase (MMP) modulator in combination with at least one pharmaceutically active agent selected from the group consisting of:
(a) cell cycle modulators;
(b) inflammatory event modulators;
(c) angiogenesis event modulators;
(d) fibroblast migration agents;
(e) fibroblast proliferation agents;
(f) tissue remodeling correcting agents;
(g) antimicrobial agents;
(h) modulators of deposition of extra cellular matrix;
(i) penetration enhancers;
(j) antioxidants;
(k) antipuritic agents;
(l) fibrinolytic agents;
(j) immunomodulators;
(m) transcription modulating agents;
(n) surface modulating agents;
(o) growth factor inhibitors; and
(p) antiproliferative agents,
wherein said medical device is adapted for implantation or insertion in the coronary vasculature, peripheral vasculature, esophagus, colon, biliary tract, brain or liver of a patient.

3. The composition of claim 1, further comprising a pharmaceutically acceptable carrier, wherein the pharmaceutically acceptable carrier comprises a blend of polyethylene glycols having different molecular weights.

4. The composition of claim 1, wherein the closed wound is a scar selected from the group consisting of: a normal scar; a hypertrophic scar; a keloid scar; a Dupuytren's contracture; a Peyronnie's Disease; a reactive scar; an excessive post-operative scar; or a fibrotic scar.

5. The composition of claim 1, wherein the closed wound is selected from a group consisting of: a wound caused by laceration; a wound caused by avulsion; a wound caused by burn; a wound caused by radiation; a wound caused by chemical facial peel; and a wound caused by accident.

6. The composition of claim 3, further comprising an anti-irritant.

7. The composition of claim 6, wherein the anti-irritant is selected from the group consisting of glycerol monooleate, diphenhydramine, calamine and C₃-C₄ diol.

8. The composition of claim 3, further comprising a deodorant agent.

9. The composition of claim 8, wherein the deodorant agent is selected from the group consisting of aluminum zirconium trichlorohydrex and zinc acetate.

10. A method of reducing the size or improving the appearance of a closed wound comprising administering the composition of claim 1.

11. The method of claim 10, wherein the combination is administered in a sequential manner.

12. The method of claim 10, wherein the combination is administered in a substantially simultaneous manner.

13. The method of claim 10, wherein the composition is topically administered to a closed wound.

14. The method of claim 10, wherein the closed wound is a scar selected from the group consisting of: a normal scar; a hypertrophic scar; a keloid scar; a Dupuytren's contracture; a Peyronnie's Disease; a reactive scar; an excessive post-operative scar; and a fibrotic scar.

15. The method of claim 10, wherein the closed wound is selected from a group consisting of a wound caused by laceration; a wound caused by avulsion; a wound caused by burn; a wound caused by radiation; a wound caused by chemical facial peel; and a wound caused by accident.
